(19) United States
(12) Patent Application Publication
(10) Pub. No.: US 2018/0052168 A1
(43) Pub. Date: Feb. 22, 2018

(54) EX VIVO LIQUID BIOPSY

(71) Applicant: HELOMICS CORPORATION, Pittsburgh, PA (US)

(72) Inventors: Neil J. CAMPBELL, Damascus, MD (US); Donald L. VERY, JR., Little Silver, NJ (US); Nicholas J. REITZE, Bethel Park, PA (US); Aaron W. GILKEY, Pittsburgh, PA (US); Randy L. SWACKHAMMER, JR., Pittsburgh, PA (US); Courtney M. DRAY, Pittsburgh, PA (US); Benjamin S. SKOFF, Homer City, PA (US); Katelynn J. THIEL, Greensburg, PA (US)

(21) Appl. No.: 15/455,568

(22) Filed: Mar. 10, 2017

Related U.S. Application Data
(60) Provisional application No. 62/306,864, filed on Mar. 11, 2016.

(51) Int. Cl. G01N 33/574

(52) U.S. Cl. CPC G01N 33/57492; G01N 33/5082; G01N 33/5748; G01N 33/57496

(57) ABSTRACT

Embodiments disclosed herein provide in vitro methods for use in the culture and biochemical analysis of normal or malignant cells in autologous, allogeneic, and xenogeneic fluids and the biomarkers secreted from those cells over time and in response to various changing conditions. The methods disclosed herein provide a clinical diagnostic and research platform that provides for the study of fundamental mechanisms of cellular morphology, cellular growth kinetics, intracellular and intercellular oncogenesis, therapeutic drug mechanisms or action and efficacy, identification and use of novel biomarkers or biomarker panels.

### RELATED APPLICATIONS AND INCORPORATION BY REFERENCE

This application claims priority to U.S. Provisional Application No. 62/306,864, filed on Mar. 11, 2016, the disclosures of which are incorporated by reference in their entirety.

### TECHNICAL FIELD

The subject matter disclosed herein is directed to in vitro methods for culturing, challenging, and measuring the response of cells to the particular challenge. Specifically, the methods disclosed herein are directed to challenging and measuring cell samples cultured in autologous, allogeneic, and xenogeneic fluids.

### BACKGROUND

A liquid biopsy, the collection of blood and/or urine from a cancer patient with primary or recurrent disease and the analysis of cancer-associated biomarkers in the blood and/or urine, is increasingly being recognized as a viable, noninvasive method of monitoring a patient's disease progression, regression, recurrence, and/or response to treatment. Numerous studies have reviewed the clinical utility of the quantitation of circulating tumor cells (CTCs; 1-6), exosomes (2, 4); and proteins (1); and the quantitation and sequencing of circulating cell-free tumor RNA (ctRNA; 1-4) and DNA (ctDNA; 1-4, 6, 7), and as diagnostic indicators of disease status or treatment efficacy.

In terms of patient morbidities, the use of a minimally-invasive, vascular, needle-stick, and/or obtaining a minimally-invasive urine specimen to obtain clinically relevant information regarding a patient's tumor, is clearly superior to the conventional, more invasive methods (large bore needle biopsy or surgical resection of the tumor) of tumor sampling. Additionally, during primary disease treatment or disease recurrence, it is often very difficult to obtain another biopsy of the tumor without interruption of treatment or significant risk to the patient. In these instances, a liquid biopsy would be very useful.

However, there are significant technical challenges in the identification and quantitation of CTCs, ctRNA, ctDNA, and exosomes in the blood and/or urine of cancer patients. These potential cancer biomarkers are generally produced by the growing tumor in very small quantities and are secreted into a large volume of circulating blood and/or excreted into the urine. As such, their quantitation usually involves the use of complex purification and concentration procedures (1). Additionally, it is not yet a generally-accepted medical fact that CTCs, ctDNA, ctRNA, or exosomes found in the blood and/or urine are representative of the growing tumor and that analysis of these biomarkers would yield clinically relevant insights regarding the heterogeneous tumor (1).

Thus, there remains a need in the medical diagnostic art for an in vitro method for use in the culture and biochemical analysis of normal or malignant cells and the biomarkers secreted from these cells with time in culture or drug treatment.

In certain example embodiments, the present invention comprises culturing human cells, animal cells, and/or cell lines in a culture medium comprising autologous, allogeneic, and/or xenogeneic fluids.

### SUMMARY

A method for ex vivo liquid biopsy processing comprises culturing ex vivo cells obtained from a tumor biopsy sample (both malignant and non-malignant stromal cells) from a subject in a culture medium comprising one or more autologous, allogeneic, and/or xenogeneic fluids, challenging the culture cells, and measuring a response to the cells. The culture cells may comprise a combination of tumor and stromal cells. The fluids that promote tumor and stromal cell growth may comprise one or more of blood, serum, ascites fluid, urine, or saliva taken from the same subject as the tumor biopsy sample or pooled from multiple donors of the same species or pooled from members of a different mammalian species.

In certain example embodiments, the culture medium may further comprise one or more of fetal calf or bovine serum, pooled human serum (from normal and/or cancer patients), and one or more purified or recombinant human growth factors.

In certain example embodiments, the method may further comprise measuring the degree of malignant cells present in a sample, for example, by immunofluorescent staining of cell surface cancer markers, or other similar method.

The cultured cells may be challenged by exposing the cells to varying types, concentrations, and durations of exposure to therapeutic agents and/or metabolic products. The cultured cells may also be challenged by exposure to different environmental conditions such as changes in exposure to different culture media additives, changes in pH, changes in atmospheric pressure, or changes in atmospheric oxygen and/or carbon dioxide concentrations, or a combination thereof.

Cell responses may be measured biochemically or optically and include, but are not limited to, cellular growth kinetics and doubling time, cell surface receptor agonism or antagonism by large and/or small molecules, signal transduction phosphorylation events, signaling pathway analyses, changes in gene expression profiles or signatures, single nucleotide polymorphism genotyping, gene structural variant detection, protein expression profiles or signatures, protein-protein or other biomolecular ligand-ligand interactions, tumor shedding profiles, changes in cell motility, morphology, cell-cell contact, spatial distribution, cell death, or a combination thereof.

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

FIG. 1 provides a schematic overview of an ex vivo liquid biopsy cell culture process for the evaluation of cellular biochemical processes, in accordance with certain example embodiments.

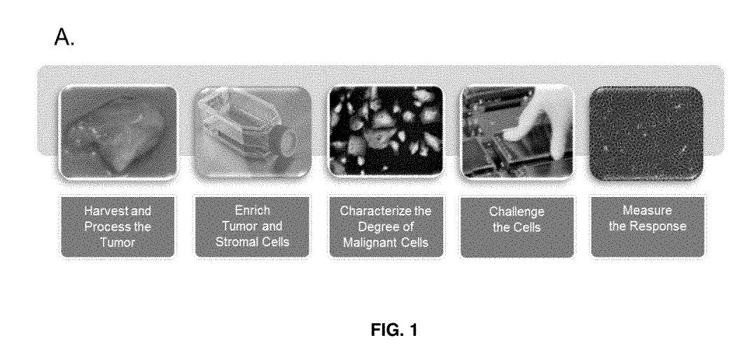

FIG. 2 provides an alternative schematic overview of an ex vivo liquid biopsy cell culture process for the evaluation of the secretion of ctDNA, ctRNA, proteins and extracelluar vesicles, in accordance with certain example embodiments.

FIG. 3 is a graph showing differential cytokine expression from serous ovarian adenocarcinoma cells derived from patient C12-02 and cultured fetal bovine serum (FBS) and pooled human serum (PHS), in accordance with certain example embodiments.

FIG. 4 is a graph showing differential cytokine expression from serous ovarian adenocarcinoma cells derived from a patient and cultured in FBS and PHS, in accordance with certain example embodiments.

FIG. 5 is a graph showing differential cytokine expression from serous ovarian adenocarcinoma cells derived two patients (C12-02 and C12-03) and cultured in pooled human serum, in accordance with certain example embodiments.

FIG. 6 is a graph showing differential cytokine expression from serous ovarian adenocarcinoma cells derived from two patients (C12-02 and C12-03) and cultured in FBS or PHS, in accordance with certain example embodiments.

FIG. 7 is a table showing the purification of ctDNA from a primary serous ovarian adenocarcinoma tumor growing in conditioned, unconditioned, and serum-free tissue culture media for 48 hours.

FIG. 8 is a Western Blot of CD-9 expression, a protein biomarker present in exosomes, showing purification of exosomes using three different commercially available exosome purification kits C12-10B1, C12-10B2, and C12-10B3.

FIG. 9 is a table showing quantitation of DNA purified, using three different commercially available exosome purification kits C12-10B1, C12-10B2, and C12-10B3, from the exosomes of a primary serous ovarian adenocarcinoma tumor grown in in vitro tissue culture.

FIG. 10 is a table showing qPCR amplification of tumor DNA (PIK3CA gene) derived from the exosomes of a primary serous ovarian adenocarcinoma tumor grown in in vitro tissue culture. (Positive amplification cutoff, Ct≦26.0).

### DETAILED DESCRIPTION OF THE EXAMPLE EMBODIMENTS

Embodiments disclosed herein provide in vitro methods for use in the culture of normal or malignant cells in autologous, allogeneic, or xenogeneic fluids and the biochemical analysis of biomarkers secreted from those cells over time and in response to various changing conditions. The methods disclosed herein provide a clinical diagnostic and research platform that provides for the study of fundamental mechanisms of cellular morphology, cellular growth kinetics, intracellular and intercellular oncogenesis, therapeutic drug mechanisms or action and efficacy, and identification and use of novel biomarkers or biomarker panels. While not limited to the following theory, it is believed that culturing of cells according to the methods disclosed herein more accurately replicates the expression of various genes, gene regulatory elements, and proteins including secreted and cell surface markers found in vivo, therefore, providing a more accurate assessment of a given cell's biology under a set of experimental conditions.

The methods disclosed herein may be used with a wide range of detection technologies to characterize the genetic and phenotypic responses of a cultured cell or cell population. For example, gene expression may be assessed using a wide variety of tools such as, but not limited to, PCR, qPCR, microarrays, and next generation sequencing technologies. Likewise, the proteome of the cultured cells may be assessed by methods such as, but not limited to, immunoassays, protein arrays, HPLC, and mass spectroscopy, or any other method capable of identifying the character of expressed proteins in a sample. In addition, the methods disclosed herein provide a process to directly monitor shedding from normal, transformed, or malignant cells and to clinically correlate the presence and clinical utility of circulating tumor cells (CTCs), circulating cell-free tumor RNA (ctRNA), circulating cell-free DNA (ctDNA), proteins, and exosomes in culture media and patient biological samples such as blood and urine.

The methods disclosed herein comprise culturing cells in culture medium comprising autologous, allogeneic, and xenogeneic fluids. The cultured cells are then challenged and a response to the challenge measured. In certain example embodiments, the cells are isolated from a biological sample of a subject. The source of the cells depends on the nature of the diagnostic or research function of the method. In certain example embodiments, the sample is a biopsy sample from a patient with a type of cancer. The method may be used to assess all types of tumor cells, including both solid and non-solid tumor cells. In certain example embodiments, only tumor cells are isolated and cultured. In certain other example embodiments, both tumor and stromal cells are isolated and cultured.

Autologous fluids refer to fluids obtained from the same subject from which the biological sample is obtained. Allogeneic fluids refers to fluid derived and pooled from members of the same species. Xenogeneic fluids refers to fluids derived and pooled from members of a different species. Autologous, allogeneic, and xenogeneic fluids include blood, serum, plasma, saliva, ascites fluid, peritoneal fluid, and urine. In certain example embodiments, the fluid is blood. In another example embodiment, the fluid is serum. In another example embodiment, the fluid is plasma. In another example embodiment, the fluid is saliva. In another example embodiment, the fluid is ascites fluid. In another example embodiment, the fluid is peritoneal fluid. In another example embodiment, the fluid is urine.

In certain example embodiments, the culture medium comprises 1% to 50%, 1% to 45%, 1% to 40%, 1% to 35%, 1% to 30%, 1% to 25%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 5% to 10%, 6% to 10%, 7% to 10%, 8% to 10%, 9% to 10%, 10% to 15%, 11% to 15%, 12% to 15%, 13% to 15%, 14% to 15%, 15% to 20%, 16% to 20%, 17% to 20%, 18% to 20%, 19% to 20%, 20% to 25%, 21% to 25%, 22% to 25%, 23% to 25%, 24% to 25%, 25% to 30%, 26% to 30%, 27% to 30%, 28% to 30%, 29% to 30%, 30% to 35%, 31% to 35%, 32% to 35%, 33% to 35%, 34% to 35%, 35% to 40%, 36% to 40%, 37% to 40%, 38% to 40%, 39% to 40%, 40% to 45%, 41% to 45%, 42% to 45%, 43% to 45%, 44% to 45%, 45% to 50%, 46% to 50%, 47% to 50%, 48% to 50%, 49% to 50%, 10% to 50%, 15% to 50%, 20% to 50%, 25% to 50%, 30% to 50%, 35% to 50%, 40% to 50%, 5% to 25%, 5% to 20%, or 5% to 15% (v/v) autologous, allogeneic, or xenogeneic fluid.

In certain example embodiments, the culture medium may further comprise one or more of fetal calf or bovine serum, pooled human serum, and purified and/or recombinant human growth factors.

The pooled human serum may be from a normal subject, a diseased subject, or combination of both. In certain example embodiments, the pooled human serum is pooled from normal subject serum. In certain other example embodiments, the pooled human serum is from cancer patient serum. In certain other example embodiments, the pooled human serum is from a combination of both normal and cancer patient serum. In certain example embodiments, the culture medium comprises 1% to 50%, 1% to 45%, 1% to 40%, 1% to 35%, 1% to 30%, 1% to 25%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 5% to 10%, 6% to 10%, 7% to 10%, 8% to 10%, 9% to 10%, 10% to 15%, 11% to 15%, 12% to 15%, 13% to 15%, 14% to 15%, 15% to 20%, 16% to 20%, 17% to 20%, 18% to 20%, 19% to 20%, 20% to 25%, 21% to 25%, 22% to 25%, 23% to 25%, 24% to 25%, 25% to 30%, 26% to 30%, 27% to 30%, 28% to 30%, 29% to 30%, 30% to 35%, 31% to 35%, 32% to 35%, 33% to 35%, 34% to 35%, 35% to 40%, 36% to 40%, 37% to 40%, 38% to 40%, 39% to 40%, 40% to 45%, 41% to 45%, 42% to 45%, 43% to 45%, 44% to 45%, 45% to 50%, 46% to 50%, 47% to 50%, 48% to 50%, 49% to 50%, 10% to 50%, 15% to 50%, 20% to 50%, 25% to 50%, 30% to 50%, 35% to 50%, 40% to 50%, 5% to 25%, 5% to 20%, or 5% to 15% (v/v) autologous, allogeneic, or xenogeneic fluid. The methods disclosed herein are not necessarily limited to cells of human origin. Therefore, one of ordinary skill in the art will recognize that when cells from another species are assessed it may be necessary to use pooled serum from that species in place of pooled human serum. The ratio of normal pooled serum to cancer pooled serum may ranged from 1:10 to 10:1.

The fetal calf or bovine serum may be sourced from any commercially available source of cell culture grade fetal calf or bovine serum. In certain example embodiments, the fetal calf or bovine serum is provided at a concentration of 1% to 50%, 1% to 45%, 1% to 40%, 1% to 35%, 1% to 30%, 1% to 25%, 1% to 20%, 1% to 19%, 1% to 18%, 1% to 17%, 1% to 16%, 1% to 15%, 1% to 14%, 1% to 13%, 1% to 12%, 1% to 11%, 1% to 10%, 1% to 9%, 1% to 8%, 1% to 7%, 1% to 6%, 1% to 5%, 1% to 4%, 1% to 3%, 1% to 2%, 5% to 10%, 6% to 10%, 7% to 10%, 8% to 10%, 9% to 10%, 10% to 15%, 11% to 15%, 12% to 15%, 13% to 15%, 14% to 15%, 15% to 20%, 16% to 20%, 17% to 20%, 18% to 20%, 19% to 20%, 20% to 25%, 21% to 25%, 22% to 25%, 23% to 25%, 24% to 25%, 25% to 30%, 26% to 30%, 27% to 30%, 28% to 30%, 29% to 30%, 30% to 35%, 31% to 35%, 32% to 35%, 33% to 35%, 34% to 35%, 35% to 40%, 36% to 40%, 37% to 40%, 38% to 40%, 39% to 40%, 40% to 45%, 41% to 45%, 42% to 45%, 43% to 45%, 44% to 45%, 45% to 50%, 46% to 50%, 47% to 50%, 48% to 50%, 49% to 50%, 10% to 50%, 15% to 50%, 20% to 50%, 25% to 50%, 30% to 50%, 35% to 50%, 40% to 50%, 5% to 25%, 5% to 20%, or 5% to 15%.

The purified or recombinant human growth factors may comprise one or more of CSF-1, CSF-2, EGF, FGF, IGF-1, IGF-2, IL-2, IL-3, IL-4, IL-6, IL-8, IL-10, IL-12, IL-13, PDFG, TGF-alpha, TGF-beta, and VEGF. In certain example embodiments, the culture medium comprises only purified human growth factors. In another example embodiment, the culture medium comprises only recombinant human growth factors. In certain other example embodiments, the culture medium comprises a combination of purified and recombinant human growth factors. In certain example embodiments, the culture medium may comprise at least 10 pg/mL, 20 pg/mL, 30 pg/mL, 40 pg/mL, 50 pg/mL, 60 pg/mL, 65 pg/mL, 70 pg/mL, 80 pg/mL, 90 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 2 ng/mL, 3 ng/mL, 4 ng/mL, 5 ng/mL, 6 ng/mL, 7 ng/mL, 8 ng/mL, 9 ng/mL or 10 ng/mL of human purified and/or recombinant human growth factors. As noted above regarding pooled serum, species specific growth factors may be required for cells isolated from other species.

In certain example embodiments, the culture medium comprises 5% to 50% (v/v) of fetal calf or bovine serum, 1% to 50% pooled human serum, 1% to 50% autologous fluids, and purified or recombinant human growth factors at a concentration of at least 10 pg/mL up to 10 ng/mL.

Cells to be cultured and assessed using the methods disclosed herein may be obtained from a variety of sources. In certain example embodiments, cells are obtained from patient biopsy samples or from tumors removed in debulking surgeries. For example, solid tumors may be collected from debulking surgeries for primary or recurrent tumors. Core needle biopsies and fine needle aspirates may be collected for the purposes of initial cancer diagnosis or the detection/diagnosis of a recurrent cancer type. “Liquid” tumors, such as leukemia or lymphoma, may be purified and obtained using standard procedures for whole blood or other clinical specimens, for example ascites obtained during paracentesis.

Samples may be processed for cell culture using known methods in the art. Example processing methods are disclosed in U.S. Pat. Nos. 8,236,489; 8,183,009; 7,771,963; 5,728,541; and 6,900,027, which are incorporated in their entirety herein by reference.

The cells are then cultured in individual discrete volumes. The individual discrete volumes may include individual cell culture flask, individual wells of a microwell plate, such as a 6, 24, 96, and 384, 1536 microwell plates, or individual droplets generated on a microfluidic cell culture device. In certain example embodiments, each individual discrete volume is exposed to a unique set of culture conditions (“challenge”) such as exposure to different types, concentrations and durations of certain therapeutic agents, exposure over varying concentrations and durations of different metabolic products of biochemical metabolism. The drugs may include small molecules (<1000 Da) and large molecules, such as but not limited to, antibodies and proteins, and immune effector cell populations. The cells may also be challenged by exposure to different environmental conditions such as, but not limited to, temperature, atmospheric pressure, atmospheric CO₂ concentration, and atmospheric O₂ at different concentrations and/or durations of exposure. The cells may also be challenged by exposure to different growth media, for example, containing different additives at varying concentrations and/or durations of exposure, and varying pH levels and/or varying durations of exposure. In certain example embodiments, the cells are cultured in replicate.

In certain example embodiments, the degree of malignant cells in each individual discrete volume may be categorized prior to challenging the cells and measuring the corresponding response. This may be accomplished, for example, using an immunofluorescent antibody assay that stains the cells for the presence of protein biomarkers present on malignant and non-malignant cells.

A response of the cells to any or a combination of the above challenges is then measured. The specific types of response to be measured will depend on the specific experiment and the specific biochemical pathway or process under investigation. Responses that may be monitored include, but are not limited to, cellular growth kinetics and doubling time, cell surface receptor agonism or antagonism by large and/or small molecules, signal transduction phosphorylation events and signaling pathway analyses, gene expression profiles or signatures, single nucleotide polymorphism genotyping, gene structural variant detection, protein expression profiles or signatures, protein-protein and other biomolecular ligand-ligand interactions. The measured response may also be phenotypic in nature and include assessments of changes in motility, morphology, cell-cell contact, spatial distribution, cell death, or a combination thereof. In certain example embodiments, phenotypic changes are assessed optically, including but not limited to use of phase contrast and fluorescence microscopy. In certain example embodiments, the phenotypic response is measured using digital pathology analyses. Digital pathology analysis may include identification of specific biomarker expression by IHC or ICC, tumor proliferation indices, stromal characterization, nuclear characterization of malignant cells, intra-tumor heterogeneity, and degree of tumor angiogenesis.

In certain example embodiments, the response is measured on the cells directly. For example, the cells may be lysed to release cellular contents in order to assess changes, for example, changes in gene expression. In certain other example embodiments, the cell culture supernatant may be collected and one or more biomarkers measured from the cell supernatant. In certain example embodiments, secretion of circulating cell-free tumor RNA, circulating cell-free tumor DNA, proteins, and extracellular vesicles, such as exosomes and microvesicles, or a combination thereof, may be isolated and assessed using the methods of the present invention.

In certain example embodiments, tissue culture media containing one or more, but not limited to FBS, PHS, autologous cancer patient serum, autologous cancer ascites fluid, and/or purified human growth factors, is used to support the growth and proliferation of patient tumor explants (both malignant cells and supportive stromal cells) ex vivo. This culture system can be used as a model to understand the interactions of malignant cells and supportive stromal cells (conscriptor cells, passenger cells or bystander cells), discover and evaluate new therapeutic drugs and/or drug targets, reevaluate existing therapeutic drugs and/or drug targets for additional mechanisms or greater understanding (drug rescue), identify new drug companion biomarkers or diagnostic biomarkers of disease, and validate the clinical utility of blood-based and/or urine-based liquid biopsy biomarkers.

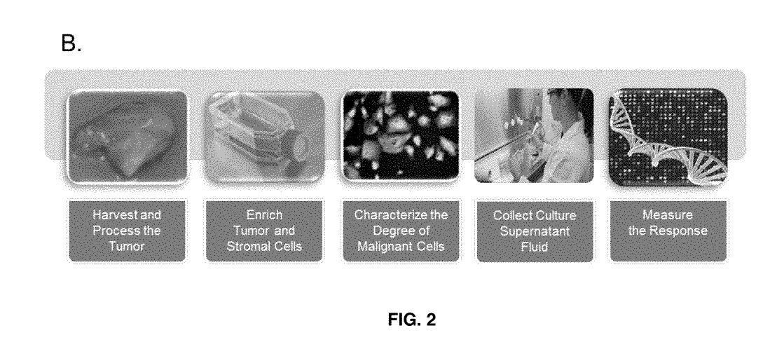

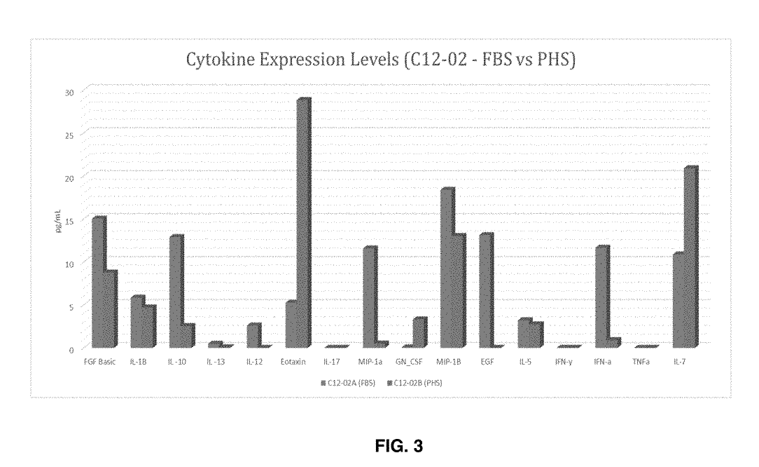

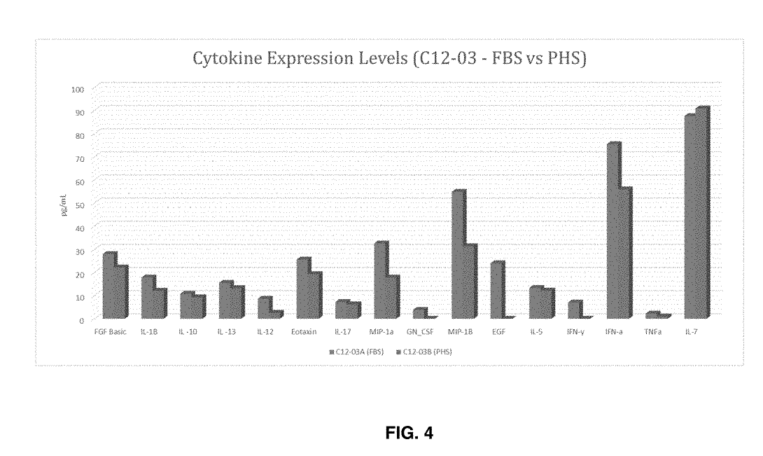

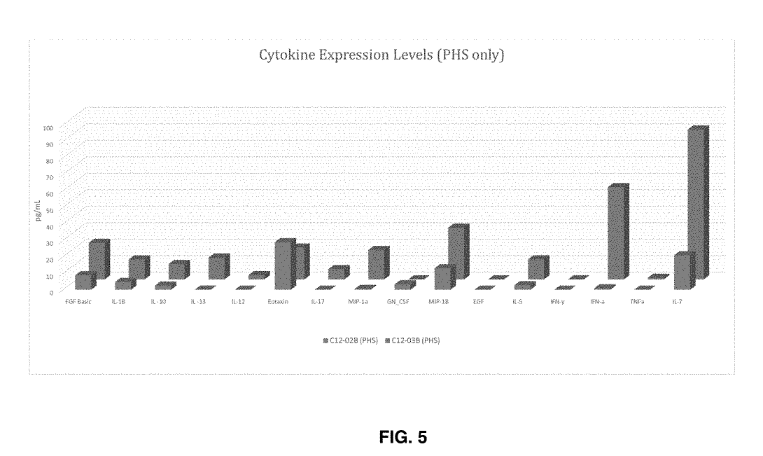

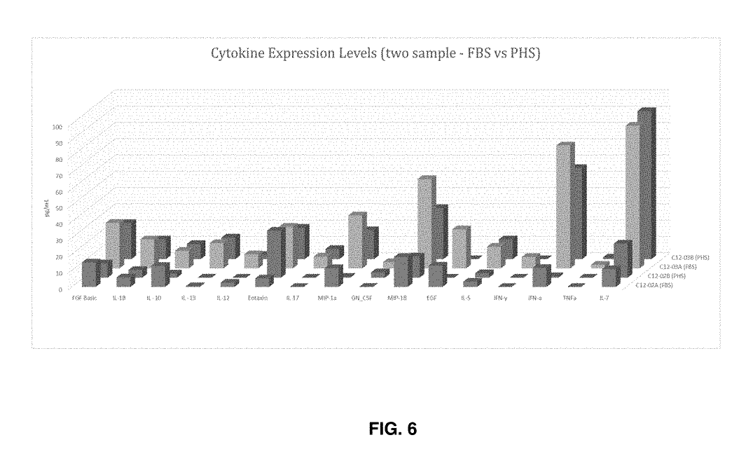

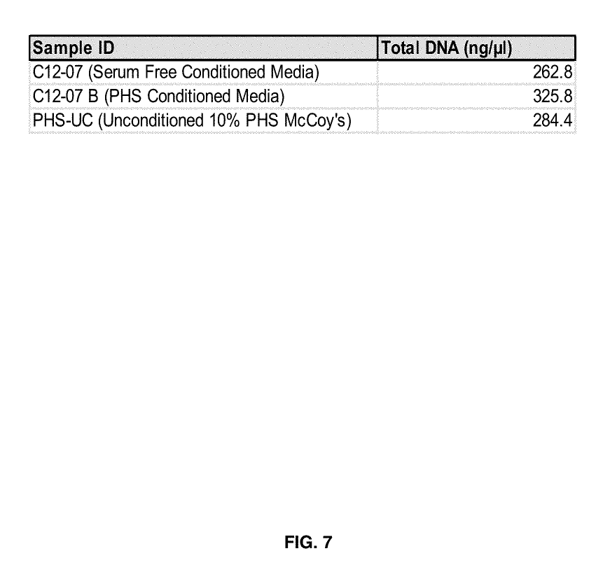

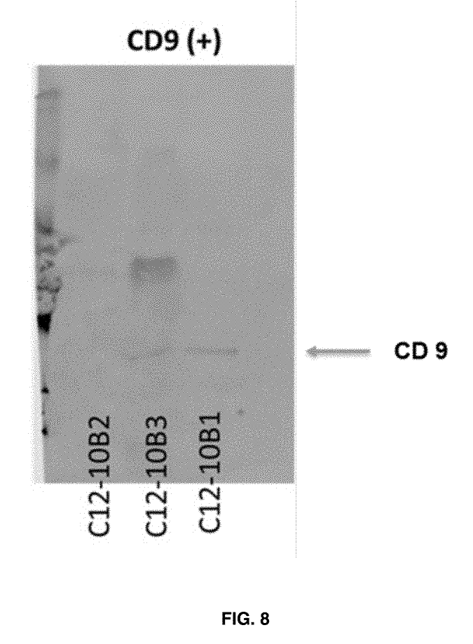

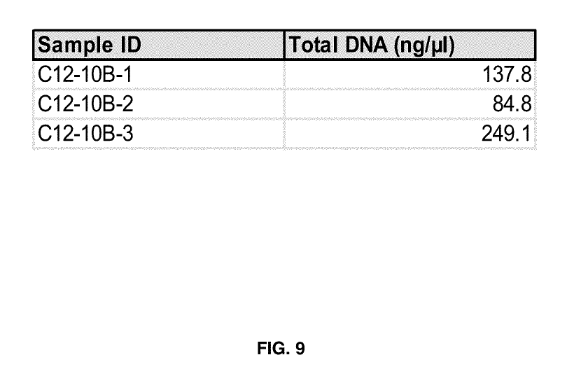

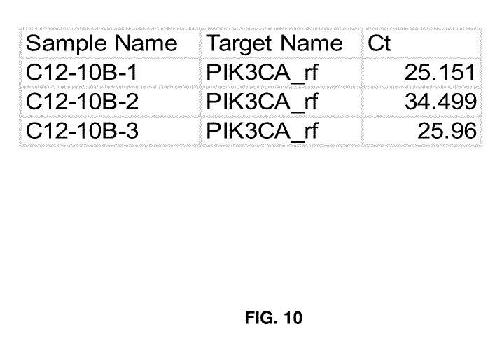

In certain other example embodiments, the methods disclosed herein are used directly to demonstrate the shedding of biomolecules and extracellular vesicles from cultured tumor cells and to develop a “shedding profile” of biomolecules for cancer patients. As shown in FIGS. 2 and 3, cancer patients exhibit a unique and personalized profile of tumor shed proteins into the cell culture supernatant fluid when patient cells are grown in tissue culture media supplemented with FBS or pooled human serum. The protein profiles are also different between different patients when their tumor cells are grown in PHS (see FIG. 4). The combined results of these protein cell shedding experiments are summarized in FIGS. 5 and 6. FIG. 7 illustrates the purification of cell-free DNA from tumor culture supernatant fluids. FIG. 8 shows the purification of CD 9-positive exosomes from tumor culture supernatant fluids. The successful purification of DNA from exosomes in tumor tissue culture supernatant fluids is shown in FIG. 9. The presence of tumor DNA in exosomes isolated from tissue culture supernatant fluids is demonstrated in FIG. 10 by the successful qPCR amplification of the PIK3CA gene.

Various modifications and variations of the described methods, pharmaceutical compositions, and kits of the disclosure will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

All publications, patents, and patent applications mentioned herein are incorporated by reference to the same extent as if each individual publication, patent or patent application was specifically and individually indicated to be incorporated by reference in its entirety. In the event of there being a difference between definitions set forth in this application and those in documents incorporated herein by reference, the definitions set forth herein control.

### REFERENCES CITED

1. Cree I A. Liquid biopsy for cancer patients: principles and practice. Pathogenesis 2: 1-4 (2015).
2. Brock G, Castellanos-Rizaldos E, Hu L, Coticchia C, and Skog J. Liquid biopsy for cancer screening, patient stratification and monitoring. Transl Cancer Res 4: 280-290 (2015). Doi: 10.3978/j.issn.2218-676X.2015.06.05
3. Karachaliou N, Mayo-de-las-Casas C, Monila-Vila M A, and Rosell R. Real-time liquid biopsies become a reality in cancer treatment. Ann Transl Med 3: 36-38 (2015). Doi: 10.3978/j.issn.2305-5839.2015.01.16.
4. Yang J. Liquid biopsy: the future work for the clinical pathologist. Austin J Clin Pathol 2: 1034-1037 (2015).
5. Alix-Panabieres C, and Pantel K. Circulating tumor cells: liquid biopsy of cancer. Clin Chem 59: 110-118 (2013).
6. Pantel K, and Alix-Panabieres C. Real-time liquid biopsy in cancer patients: fact or fiction? Cancer Res 73 (2013). Doi: 10.1158/008-5274.CAN-13-2030.
7. Heitzer E, and Geigl J. Circulating tumor DNA as a liquid biopsy for cancer. Clin Chem 61: 112-113 (2015).

What is claimed is:

1. A method of ex vivo liquid biopsy processing, comprising:
culturing, in vitro, cells obtained from a tumor biopsy sample from a subject in a culture medium comprising an autologous, allogeneic, or xenogeneic fluid;
challenging the cultured cells; and
measuring a response of the cultured cells.

2. The method of claim 1, wherein the culture medium comprises an autologous fluid from the subject.

3. The method of claim 1, wherein the cells comprise tumor cells and stromal cells.

4. The method of claim 1, wherein the culture medium further comprises one or more of fetal calf or bovine serum, pooled human serum, and human growth factors.

5. The method of claim 4, wherein the pooled human serum is pooled only from normal patients, only from cancer patients, or is pooled from both normal and cancer patients.

6. The method of claim 4, wherein the human growth factors are purified human growth factors, recombinant human growth factors, or a combination thereof.

7. The method of claim 4, wherein the culture medium comprises 1% to 50% (v/v) of autologous fluid, 1% to 50% fetal calf or bovine serum, and up to 10 ng/mL of human growth factors.

8. The method of claim 1, further comprising measuring the degree of malignant cells present in the cultured cells.

9. The method of claim 1, wherein the response is measured biochemically, optically, or a combination thereof.

10. The method of claim 9, wherein the response measured biochemically comprises one or more of cellular growth kinetics and doubling time, cell surface receptor agonism or antagonism by large and/or small molecules, signal transduction phosphorylation events, signaling pathway analyses, changes in gene expression profiles or signatures, single nucleotide polymorphism genotyping, gene structural variant detection, protein expression profiles or signatures, protein-protein or other biomolecular ligand-ligand interactions, or tumor shedding profiles.

11. The method of claim 10, wherein the tumor shedding profiles comprises sampling the cell culture supernatant and detecting one or more of ctRNA, ctDNA, proteins, and tumor extracellular vesicles and their biochemical contents.

12. The method of claim 11, wherein tumor extracellular vesicles comprises exosomes and microvesicles.

13. The method of claim 1, wherein the tumor biopsy sample is a solid tumor biopsy sample.

14. The method of claim 1, wherein the tumor biopsy sample is obtained from blood, saliva, or urine.

15. The method of claim 1, wherein the tumor biopsy sample is a colon cancer biopsy sample, a lung cancer biopsy sample, a gynecological cancer biopsy sample, a breast cancer biopsy sample, a prostate cancer biopsy sample, a brain cancer biopsy sample, a bone cancer biopsy sample, a liver cancer biopsy sample, a bladder cancer biopsy sample, a kidney cancer biopsy sample, a rectal cancer biopsy sample, a stomach cancer biopsy sample, a leukemia biopsy sample, or a lymphoma biopsy sample.

16. The method of claim 1, wherein challenging the cultured cells comprises exposing the cells to one or more therapeutic agents, exposing the cells to different metabolic products of biochemical metabolism, or exposure to different environmental conditions.

17. The method of claim 16, wherein the cells are cultured in replicates in separate discrete volumes, and wherein of the separate discrete volumes each individual discrete volume receives a different set of challenge conditions.

18. The method of claim 16, wherein exposing the cells to one or more therapeutic agents comprises exposing different individual discrete volumes to different types and/or concentrations of therapeutic agents and optionally at different durations of exposure.

19. The method of claim 16, wherein exposing the cells to one or more metabolic products comprises exposing different individual discrete volumes to different types and/or concentrations of metabolic products and optionally at different durations of exposure.

20. The method of claim 16, wherein exposure to different environmental conditions comprises exposure to different growth media, for example, containing different additives at varying concentrations and/or durations of exposure, and varying pH levels and/or varying durations of exposure.

21. The method of claim 16, further comprising determining a treatment regimen for the subject by selecting the one or more therapeutic agents that demonstrate an anti-tumor response in the cultured cells.

22. The method of claim 21, wherein the anti-tumor response is measured by a decrease in cell motility, a decrease in cell adhesion, a decrease in cell growth, changes in gene expression, or an increase in apoptosis.
